(19) United States
(12) Patent Application Publication
(10) Pub. No.: US 2002/0120331 A1
(43) Pub. Date: Aug. 29, 2002

(54) Refractive anterior chamber intraocular implant

(76) Inventors: Miles A. Galin, New York, NY (US); Scott M. Hampton, Cumming, GA (US)

Correspondence Address: BAKER BOTTS L.L.P., 44TH FLOOR, 30 ROCKEFELLER PLAZA, NEW YORK, NY, 10112-0228, US

(21) Appl. No.: 09/794,990

(22) Filed: Feb. 27, 2001

(51) Int. Cl. A61F 2/16

(52) U.S. Cl. 623/6.49; 623/6.51; 623/6.54

(57) ABSTRACT

The present disclosure relates generally to a refractive anterior chamber ocular implant for placement in the anatomic angle of a phakic eye, the implant having an artificial lens or optic with an anterior surface and a posterior surface and having a haptic for engaging and positioning the optic in the anatomic angle, wherein the haptic possesses in an extension pad that extends and tapers in thickness from the haptic in an axial direction. The disclosure is further directed to methods for correcting refractive errors in a patient in need thereof having a phakic eye and an anatomic angle by surgically implanting an angle supported anterior chamber ocular implant having an optic with the desired refractive properties and a haptic having an extension pad that extends and tapers in thickness from the haptic in an axial direction, and anchoring the extension pad into the anatomic angle of the eye.

### FIELD OF THE INVENTION

The present invention relates generally to a refractive anterior chamber intraocular implant, for placement in the anatomic angle of an eye, having an optic with an anterior and posterior surface and one or more haptics for contacting the anatomic angle and positioning and maintaining the optic in the anterior chamber, wherein the one or more haptics possess an improved configuration. This improved angle fixation haptic may support any type of optic body including a negative, positive, astigmatic or multifocal power lens to correct refractive errors resulting from conditions such as myopia, hyperopia, or astigmatism. The present invention is further directed to a method for treating refractive errors in a patient in need thereof comprising surgically implanting an angle supported anterior chamber ocular implant having an optic with the desired refractive properties and one or more haptics having an improved configuration, and anchoring the haptic into the anatomic angle of the eye.

### BACKGROUND OF THE INVENTION

It is well known to those skilled in the art that intraocular lenses are predominantly designed to replace a previously or simultaneously removed human lens in a cataract patient (see, for example, U.S. Pat. No. 5,628,798). However, although the implantation of intraocular lenses has constituted an appreciable surgical advance, such implantation has been known to cause immediate or latent damage to the corneal endothelium, immediate or latent inflammatory responses in the anterior and/or posterior segments of the eye, immediate or latent secondary fibrosis and/or neovascularization, and other problems.

Intraocular lenses have been surgically implanted into an aphakic eye in order to take the place of the previously removed natural lens. (See, U.S. Pat. No. 4,240,163). Intraocular lenses have been implanted into the posterior chamber of the phakic eye, i.e., an eye having a natural lens in situ, to compensate for refractive errors or to create a specific refraction to assist in visual function. (See, U.S. Pat. No. 4,769,035).

Certain difficulties are associated with implanting an intraocular lens in the phakic eye that are encountered less frequently when implanting a lens in the aphakic eye. The phakic eye is a substantially more reactive environment than the aphakic eye. Inflammatory reactions tend to be greater in the phakic eye resulting in a concomitant increase in damage to the eye caused by implanting intraocular lenses. One reason is that in the aphakic eye, the natural lens does not pull on the highly reactive ciliary body thus, the ciliary body is in a “resting state” and tends to undergo some degree of atrophy. Additionally, the presence of the natural lens in the phakic eye crowds the area in which an intraocular implant can be placed in the eye. Further, in the aphakic state the configuration of the iris diaphragm is altered as is the angle the iris, which subtends from the ciliary body, both of which reduce tissue prosthesis contact in the aphakic state.

Placements of intraocular lenses in the posterior chamber of the phakic eye also have been known to cause cataract formation in the natural lens that remains in situ due to contact between the implant and the natural lens. In contrast, implanting intraocular lenses in patients having cataract removal cannot induce such an effect since the natural lens has been removed.

The anterior chamber of an eye is that area in front of the iris and behind the cornea. The anatomic angle resides in the region of the anterior chamber between the ciliary body, iris and the corneal endothelium. The iris acts as a divider between the anterior chamber and the posterior chamber. The anterior chamber was originally studied as a preferred location for aphakic intraocular implants particularly when no posterior capsule was present. However, significant drawbacks were discovered.

The phakic eye has a shallower anterior chamber (i.e., the average antero-posterior depth is less) than the aphakic eye, and the iris is in broad contact with the anatomic lens. Therefore, if an inflammatory reaction occurs in the phakic eye, there can be adherence of the iris to the anterior surface of the anatomic lens. Furthermore, if posterior chamber implants were to cause an inflammatory reaction in the phakic eye, cataract formation may occur in the natural lens which remains in situ.

For phakic intraocular lens implantation, it was assumed in the art that the preferred location of the implant was in the posterior chamber, i.e., that area behind the iris and in front of the natural lens in situ because that is the preferred placement in pseudophakia. Intraocular implants for the posterior chamber have been designed to treat myopia (nearsightedness) and hyperopia (farsightedness).

However, several drawbacks existed for correcting refractive errors in patients with high myopia, extreme nearsightedness, with posterior chamber ocular lenses. Because of the high degree of corrective refraction required, the optical zone is quite small, the peripheral optic is quite thick and consequently a myopic posterior chamber lens may irritate the iris peripherally as well as potentially touch the natural lens. Therefore, anterior chamber intraocular lenses for high myopia were explored for implantation in the phakic eye which would not be in contact with uveal tissue or the human lens. Hyperopic posterior lenses have broad iris contact and may cause pupillary block and may also cause contact with the human lens. Therefore, there is no incentive to place a hyperopic lens into the anterior chamber.

It has been generally acknowledged by those skilled in the art that there are significant risks involved with the use of anterior chamber angle supported implants in the aphakic eye particularly with closed loop lenses (reviewed in Apple et al., in Intraocular Lenses: Evolution, Designs, Complications and Pathology (William and Wilkens, Baltimore) 1989, Chapter 4, pp. 59-105) and more so in the phakic eye (Id. at p. 65, col. 1). For example, when presently configured angle supported implants are inserted into the eye, temporary or permanent adhesions of the implant to iris tissue may result, causing damage to these structures to ensue either immediately or over the long term affecting pupillary mobility and contour. In addition, once the implant is in position, it may cause similar angle adhesions due to mechanical and/or chemical inflammation which may lead to fibrosis of a progressive nature. Further, presently configured angle supported refractive implants may obstruct normal iris contraction causing an accordion like effect on the iris periphery as well as iris prolapse distal to haptic support resulting in a cats eye pupil. This would make subsequent removal of the implant a complex, dangerous surgical procedure. Other problems associated with such implants are cataract formation, secondary glaucoma, corneal edema, hyphema, and progressive endothelial cell loss, in addition to other complications.

Anterior chamber intraocular lenses have been designed for placement in a phakic eye, including implants disclosed in U.S. Pat. Nos. 4,676,792 and 5,071,432 and European Patent Publication No. EP 0195881, however, such implants have resulted in reported complications. For instance, according to Savagoussi et al., Refract. Corneal Surg. 7:282-285 (1991), damage to the corneal endothelium was reported after implantation of angle supported anterior chamber intraocular implants in the phakic eye making further implantation of the Baikoff ZB implant disclosed therein unacceptable.

As observed in Ophthalmology Alert, Vol. 1, No. 11 (Nov. 1990), pp. 41-42, Comment on page 42, several American manufacturing companies that were preparing to begin clinical trials of phakic anterior chamber ocular implants in the United States are now likely to abandon these studies, due to the attendant risks associated with anterior chamber implants in the phakic eye and the difficulty of obtaining approval of the U.S. Food and Drug Administration (FDA) for the use of the implants. A significant risk involved in the use of such anterior chamber implants in the phakic eye is the potential for the implanted lens to contact the corneal endothelium, the anatomic lens or the iris with resultant complications, even with the enhanced vault design theorized in the studies discussed in Ophthalmology Alert because such design would bring the lens optic edge quite close to the midperipheral corneal endothelium. One alternative to avoid contact with the corneal endothelium is to reduce the diameter of the optic of the minus power lens, however, such a modification creates significant drawbacks, including glare and haloing under low light conditions. Reducing the optic size will not reduce ovalization of the pupil, and, as reported in Perez-Santonja et al., J. Cataract Refract Surg 22:183-187 (1996) (discussing Baikoff ZB5M), it will not reduce an inflammatory response.

A wide variety of haptic designs have been developed providing a smooth and uniform contact area where the haptic engages the angle.

It would clearly be advantageous to employ a means of positioning an uncoated anterior chamber ocular implant that would avoid the occurrence of the above-described problems associated with anterior chamber implants.

### SUMMARY OF THE INVENTION

The present invention is directed to a refractive anterior chamber implant, for placement in the anatomic angle of an eye, having an optic with an anterior and posterior surface and one or more haptics for contacting the anatomic angle and positioning the optic in the anterior chamber, wherein the one or more haptics possess an improved haptic configuration. In a first non-limiting embodiment of the invention, the one or more haptics possess one or more extension pads that radially extend and taper in thickness from the haptic in a direction anterior to the surface of the optic.

In a second non-limiting embodiment of the invention, the one or more haptics possess a proximal and distal portion in relation to the optic and a transitional portion connecting the proximal and distal portions, wherein the proximal portion resides substantially within the radial plane of the optic and wherein the distal portion of the haptic resides substantially in a radial plane parallel to the radial plane of the optic.

Such improved angle fixation haptics may support any type of optic body including a negative, positive, astigmatic, or multifocal power lens to correct refractive errors resulting from conditions such as myopia, hyperopia, or astigmatism.

The invention is directed to an angle supported anterior chamber ocular implant comprising an artificial refracting lens having either a positive, negative, astigmatic, or multifocal power and means for positioning the lens in the anterior chamber, wherein the means for positioning further comprises an extension pad that radially extends and tapers in thickness in a direction anterior to the surface of the optic.

In an alternative non-limiting embodiment, the inventive implant has a special degree of flexure so that when pressure is applied to the implant upon surgical insertion to an eye, the haptics compress and the optic vaults in a manner wherein contact with the iris, cornea, and other anatomical bodies in the eye is avoided and wherein blood supply in the anatomic angle of the eye is not cut off when the haptics are implanted therein.

In other alternative non-limiting embodiments, the implant of the present invention is uncoated or coated with a medicament comprising a compatible sulfated polysaccharide as disclosed herein and as known in the art.

The present invention is further directed to methods for treating refractive errors, such as myopia, hyperopia, or astigmatism, in a patient in need thereof, comprising surgically implanting an angle supported anterior chamber ocular implant having an optic with the desired refractive properties and one or more haptics having one or more haptic extension pads that radially extend and taper in thickness from the haptic in a direction anterior to the surface of the optic, and anchoring the one or more haptic extension pad into the anatomic angle of the eye. In an alternative nonlimiting embodiment of the invention, the one or more haptic extension pad is anchored into the anatomic angle of the eye predominantly anterior to the scleral spur because of the anterior directed haptic extension pad which is anteriorly directed with respect to the iris as well.

The present invention is further directed to methods of correcting refractive errors, such as myopia, hyperopia, or astigmatism, in a patient in need thereof, comprising surgically implanting an angle supported anterior chamber ocular implant having an optic with the desired refractive properties and a haptic having a proximal portion, a distal portion and a transitional portion, wherein the proximal portion of the haptic resides substantially within the radial plane of the optic, wherein the distal portion of the haptic resides substantially in a radial plane parallel to the radial plane of the optic, wherein the transitional portion possesses a double reflex curvature, and anchoring the distal haptic into the anatomic angle of the eye. In an alternative non-limiting embodiment of the invention, the distal portion of the haptic has at least one haptic extension pad that radially extends and tapers in thickness from the haptic in a direction anterior to the surface of the optic.

### BRIEF DESCRIPTION OF THE FIGURES

FIG. 1 is a top planar view of an anterior chamber angle supported ocular implant having haptic extension pads in accordance with the present invention.

FIGS. 2A-F are a cross-sectional views of alternative embodiments of insert A of FIG. 1 along plane I.

FIG. 3A-B are side views of a minus power and positive power anterior chamber angle supported ocular implant according to the present invention, respectively.

FIG. 4 shows a partial top planar view of a number of anterior chamber angle supported ocular implants having one or more haptic extension pads in accordance with the present invention.

### DETAILED DESCRIPTION OF THE INVENTION

The invention is directed to a positive, negative, astigmatic or multifocal power anterior chamber angle supported ocular implant of a phakic eye comprising an artificial refracting lens having the desired refractive properties and means for positioning the lens in the anterior chamber angle of the eye, wherein contact between the lens and other anatomic bodies is avoided, and wherein the means for positioning avoids contact with the iris, prevents iris angle prolapse and consequential pupillary abnormalities, and mitigates damage to the anatomic angle of the eye.

As used in this description and the appended claims, the term “anterior chamber ocular implant” refers specifically to a refracting lens and means for positioning said lens in the angle which together can be surgically implanted in the phakic eye to compensate for and/or correct refractive errors and specifically excludes intraocular lenses which are surgically inserted in the aphakic eye, such as are disclosed, for example, in U.S. Pat. No. 4,240,163.

The invention is based in part on the discovery that the anterior chamber in a phakic eye of a person with hyperopia has enough room for placement of the inventive anterior chamber intraocular lens. Specifically, approximately 75% of persons with hyperopia have an anterior chamber depth of at least 2.7 to 2.8 mm.

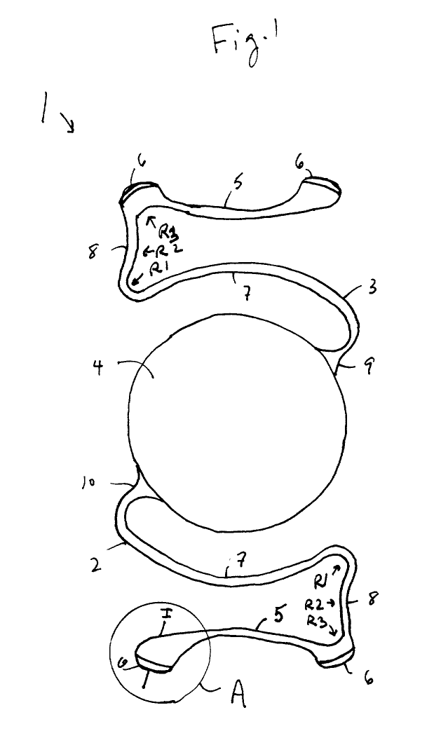

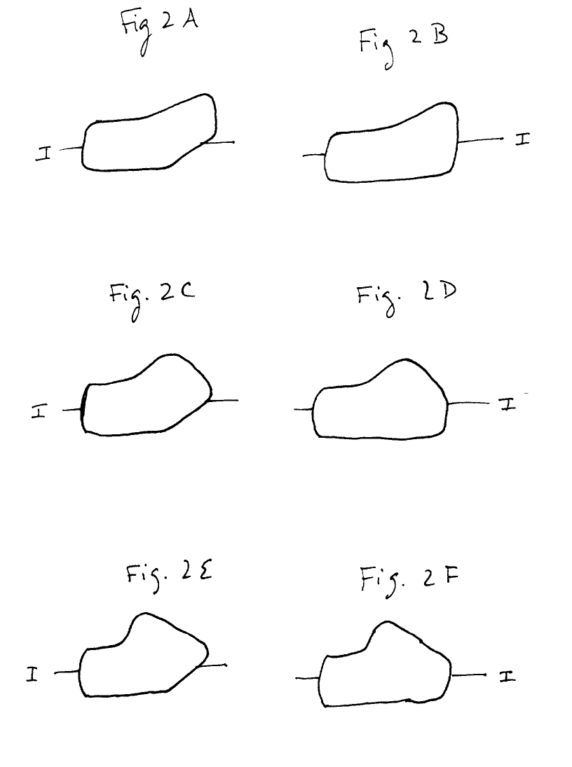

In a preferred embodiment of this invention, as depicted in FIG. 1, a preferred refractive anterior chamber ocular implant 1 has two haptics 2 and 3 integral to the refracting lens 4, so that when implanted into the anterior chamber of the eye, the lens 4 is positioned and maintained by the haptics to prevent contact between it and other anatomical bodies such as the anatomical lens, the iris, and the corneal endothelium. In addition, the distal haptics 5 and 6 are so configured as to lift off the iris plane to prevent copture of the iris distal to 5 with consequential anterior synechia formation, fibrosis and anisocoria. However, in alternative embodiments, the anterior surface and the posterior surface of the lens 4 may be concave, convex or planar, in order to achieve the desired degree of refraction.

The optical portion of the uncoated refractive implant employed in the present invention, commonly referred to as the lens or optic 4, is preferably fabricated from compounds such as polymethylmethacrylate, poly-2-hydroxyethylmethacrylate, methylmethacrylate copolymers, siloxanylalkyl, fluoroalkyl and aryl methacrylates, silicone, silicone elastomers, polysulfones, polyvinyl alcohols, polyethylene oxides, copolymers of fluoroacrylates and methacrylates, and polymers and copolymers of hydroxyalkyl methacrylates, such as 2-hydroxymethyl methacrylate, glyceryl methacrylate, 2-hydroxypropyl methacrylate, as well as methacrylic acid, acrylic acid, acrylamide methacrylamide, N,N-dimethylacrylamide, and N-vinylpyrrolidone. The artificial refracting lens 4 of the present invention may be foldable or rigid depending upon the particular selected composition of the lens.

The refracting lens of the inventive implant has a lens shape with two refractive surfaces, an anterior and posterior surface, such that the combined refractive powers of the two surfaces provides the desired degree of refraction. Lenses having at least one convex surface are typically employed to correct hyperopia. The other surface may be planar, convex or concave. In a specific non-limiting embodiment, the anterior surface is convex; and in an alternative embodiment the posterior surface is convex. Lenses having at least one concave surface are typically employed to correct myopia. Lenses may have astigmatic correction on either surface.

It is well known to those skilled in the art that when positioning an implant within the anterior chamber of the phakic eye, it is important to avoid, inter alia, contact between the implant and the anatomic lens residing in the posterior chamber as well as the iris anterior to the natural lens. In the present invention, the vault and sagitta values of the implant and means for positioning the optical portion of the implant in the anterior chamber angle of the eye to prevent such contact with other anatomic bodies during insertion and maintenance are integral. The vault is measured in relation to a flat surface upon which the haptics may rest and the posterior surface of the optic, when the implant is in a resting position or implanted in the eye. The sagitta is measured in relation to a flat surface upon which the haptics may rest and the anterior surface of the optic, when the implant is in a resting position or implanted in the eye. Displacement is the change in position of the optic from a resting position to a position in a posterior or anterior direction to the anatomic lens. Displacement of the optic from a resting position to a position anterior to the anatomic lens is known herein as positive displacement, Displacement of the optic from a resting position to a position posterior to the anatomic lens is known as negative displacement.

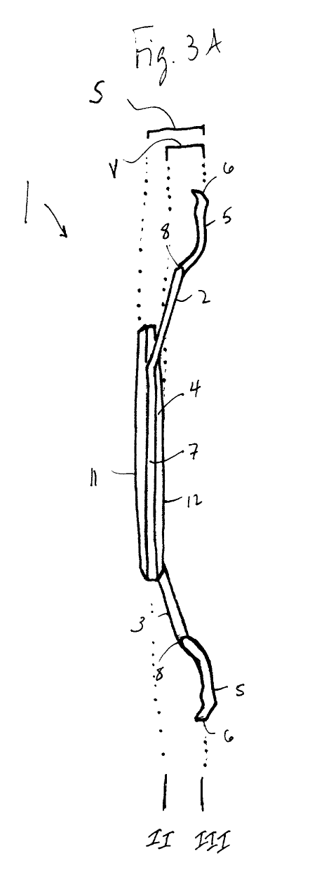

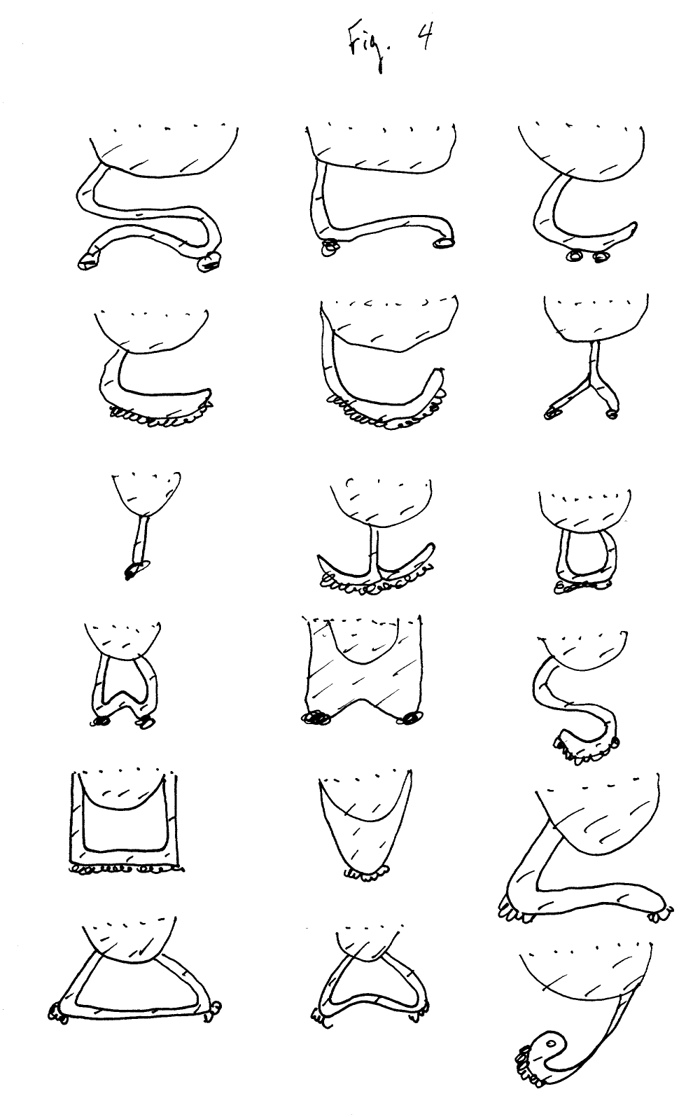

In a specific preferred embodiment, the haptics are designed having one or more extension pads that radially extend and taper in the thickness from the haptic in a direction anterior to the surface of the optic and iris to anchor the implant in the anatomic angle of the eye preferably anterior to the scleral spur and beneath Schwalbes line. According to the present invention the extension pads may be designed with a variety of configurations, as illustrated in FIG. 2. In addition, extension pads may be used with haptics of any configuration, as illustrated in FIG. 4. Having an extension pad axially extending in an anterior direction to the iris creates an anterior vector, which reduces axial displacement, leaving the iris free to dilate and constrict without restriction.

In a preferred embodiment, two haptics are connected to the optic wherein each haptic has an “S” configuration, as illustrated in FIG. 1, having a proximal portion 7, a distal portion 5 and a transitional portion 8 in relation to the optic. In the “S” configuration, the portion of the “S” configuration distal to the optic is concave in relation to the angle recess contact, is anterior to the iris plane and does not impinge posteriorly into the iris. A two-point attachment of the haptic results wherein the contact with the angle occurs at one or more extension pads 6, preferably anterior to the scleral spur, which minimizes the haptic contact with the ciliary body and iris and its consequent synechia formation and pupillary distortion. This backward and off the iris curvature S of the haptics should be within the outer flat contact plane as also seen in FIG. 1. The portion of the “S” configuration proximal to the optic 7 is convex in relation to the peripheral curvature of the optic and lies generally in the same plane as the optic anterior to the iris plane. The transition zone 8 of the haptic, which is the region between the proximal and distal portions of the haptic, causes the proximal and distal portions of the haptic to reside in separate planes in relation to the optic.

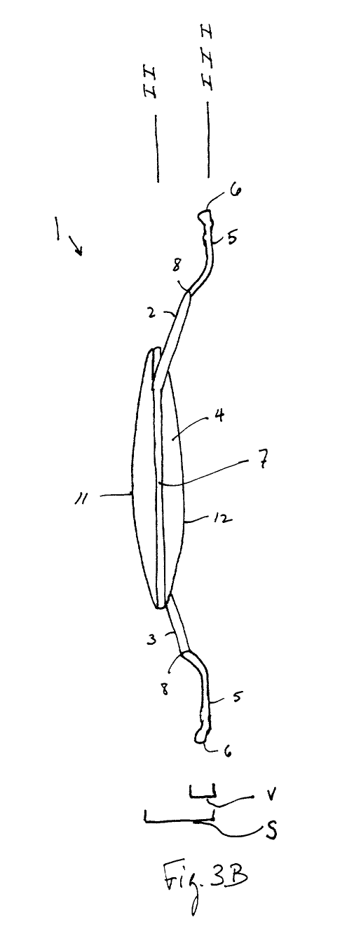

The haptics 2 and 3 suspend the lens 4 in the anterior chamber of the eye at a vault V between the range of 0.8 mm to 1.2 mm, preferably 1.0 mm, to prevent contact of the refracting lens with the natural crystalline lens or iris. A minimal sagitta S value is preferred and a minimal displacement of the optic is preferred. Maximum sagitta values range between 1.2 mm to 1.75 mm. See FIGS. 3A and 3B.

Further, the haptics preferably are made of highly flexible material having varying degrees of curvature. The haptics may be made of the same material as described above for the optical portion of the implant, or may be made of materials such as polypropylene. Depending on the composition selected, flexure of the haptics may be varied. The thickness of each haptic is greater than its width in order to reduce displacement of the implant under diametrical compression.

In a preferred embodiment, compression of the haptics is achieved through a structural design of the haptics wherein the proximal portion of the haptic 7 resides generally in the same plane II of the optic and the distal portion of the haptic 5 resides generally in a separate, generally parallel plane III in relation to the optic plane II. Planes I and II reside anterior to and generally parallel with the iris. It has been discovered that the transitional portion of the haptic, which creates the difference in plane between the proximal and distal haptic, decreases the ratio between compression and vault.

In a preferred embodiment of the invention the transition portion of this haptic possesses a change in degree of curvature from convex to concave and from concave to convex. This design has been termed a double reflex curve. As the proximal haptic transitions into the transition portion, it possesses a first degree of curvature R1, which first changes to a opposite second degree of curvature R2, and then to a third degree of curvature R3. As illustrated in FIG. 1, the first degree of curvature R1 is convex, the second degree of curvature R2 is concave, and the third degree of curvature R3 is convex. The design of the transitional portion allows for advantageously increasing the length of the proximal portion of the haptic 7 and reducing axial displacement of the optic during compression of the haptic without introducing torque. Further, the transition portion 8 dramatically reduces the compression force transmitted to the tissue in the angle, blood supply to the anatomic angle is not cut off, and subsequent necrosis of angle tissue is avoided so as not to distort iris' architecture.

The transition portion of the haptic allows the distal portion of the haptic to flex without displacing the optic, and allows the proximal haptic to achieve a maximum length without exceeding the width of incision.

In a further non-limiting embodiment of the invention, the outer radii of the contact pads are concentric with the perimeter of the optic and are designed to correspond to the contour of the anatomic angle. This design further reduces the chance of force concentrations. The contact pads may be placed in a variety of locations depending upon the kind of haptic design. Some of these varieties are illustrated in FIG. 4. Further, the way in which contact pads extend in an anterior direction may be modified as illustrated in FIG. 2, but are so vectored as to meet the angle anterior to the scleral spur in an avascular area.

In a further preferred embodiment, each haptic is designed normal to the optic 9 and 10, as shown in FIG. 1, and has no blend zone or bumps at all. The blend radius (i.e. degree of curvature) of the haptics where the haptic transitions with the optic is between 0.1 mm at a minimum and 0.4 mm, wherein a smaller blend radius is preferred. In both the horizontal plane and vertical plane, the blend radius between the optic and each haptic is preferably at right angles. However, because a right angle may not be achievable with conventional machinery, a 0.2 mm blend radius is presently the preferred working embodiment. This transition between optic and haptic maximizes the proximal haptic length, places all torques on the proximal haptic 7 on the same line as the optic 4 so that a stable couple is created reducing tilt, and markedly reduces adverse optical transients and torque/tilt transients which could result in serious damage to the eye. The transition between optic and haptic further reduces glare effects, allows for the optic diameter to be large with a maximum overall distance, and reduces optic mass.

The haptic design having a normal transition between the optic and the haptic, a long proximal haptic, and having a transitional portion of the haptic that creates a difference in plane between the proximal and distal portions of the haptic contribute to a reduced risk of endothelial cell damage, a reduced risk of damage to the natural lens during and after surgical implantation in the anterior of a phakic eye, a reduced glare and haloing, and a reduced ovaling of the pupil.

A vault value between the range of 0.8 to 1.2 with a maximum sagitta value between the range of 1.3 mm to 1.75 mm, ensures that any limited changes in vault that may occur will not inflict damage to other anatomic bodies. In a preferred embodiment, during insertion or when in position haptic compression of about 1 mm causes a vault of the optic of the anatomic lens limited to about 0.1 mm. The haptic design precludes necrosis of angle tissue and does not to distort the iris' architecture.

Refractive anterior chamber ocular implants made in accordance with the present invention have an overall omega value of 12-14 mm and an optical diameter of 5 to 7 mm. An omega value is the overall diameter of a container into which the implant may be placed. The center thickness of the optical portion of the implant may be in the approximate range of between 0.05 mm to 2.0 mm in the center of the optic and 0.1 mm to 0.8 mm around the periphery of the optic, which ranges vary with the degree of power.

In an alternative, non-limiting embodiment, the refractive anterior chamber ocular implant of the present invention may be coated. The coating may comprise any compatible sulfated polysaccharide medicament. This coating is preferably selected from the group consisting of heparin, heparin sulfate, chondroitin sulfate, dermatan sulfate; chitosan sulfate, xylan sulfate, dextran sulfate, and sulfated hyaluronic acid. The coating of the implant may be bonded to the surface of the implant by any method of bonding well known by those skilled in the art, for example, in accordance with U.S. Pat. No. 5,652,014, and preferably in such a manner that the coating is bonded to the surface of the implant by means of covalent bonding, ionic bonding, or hydrogen bonding, with covalent bonding being particularly preferred. In a preferred non-limiting embodiment, the implant surface is coated with a biocompatible polysaccharide medicament by way of end-group attachment to the implant.

Additionally, compounds which absorb ultraviolet or other short wavelength (e.g. below about 400 nm) radiation, such as compounds derived from benzotriazole groups, benzophenone groups, or mixtures thereof may be added to the monomers and/or polymers which constitute the anterior chamber ocular implant. Other compounds well known to those skilled in the art may also be used in fabricating the anterior chamber ocular implant employed in this invention.

The invention described and claimed herein is not to be limited in scope by the specific embodiments herein disclosed which are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Various references are cited herein, the disclosures of which are incorporated by reference in their entireties.

We claim:

1. A refractive anterior chamber ocular implant for placement in a phakic eye having an anatomic angle comprising
an artificial refractive lens having a radius, an anterior surface and a posterior surface; and
a haptic for positioning the implant in the anatomic angle, wherein the haptic comprises a proximal portion to the optic, a distal portion to the optic, and a transitional portion connecting the proximal and distal portions of the haptic, wherein the proximal portion resides substantially within the radial plane of the optic, wherein the distal portion of the haptic resides substantially within a radial plane parallel to the radial plane of the optic, and wherein the transitional portion comprises a first, a second and a third degree of curvature.

2. The implant according to claim 1, wherein the first and the third degrees of curvature are convex and the second degree of curvature is concave.

3. The implant according to claim 1, wherein the artificial refractive lens is positive power.

4. The implant according to claim 1, wherein the artificial refractive lens is negative power.

5. The implant according to claim 1, wherein the artificial refractive lens is astigmatic.

6. The implant according to claim 1, wherein the artificial refractive lens is multifocal.

7. The implant according to claim 1, wherein the distal portion of the haptic comprises an extension pad extending in an anterior direction and tapers in thickness from the haptic.

8. The implant according to claim 1, wherein a vault is formed between the distal portion of said haptic and the posterior surface of the artificial refractive lens and wherein the vault values range from 0.8 mm to 1.2 mm.

9. The implant according to claim 1, wherein a sagitta is formed between the distal portion of said haptic and the anterior surface of the artificial refractive lens and wherein the values range from 1.2 mm to 1.75 mm.

10. A method of correcting refractive errors in a patient in need thereof having a phakic eye and having an anatomic angle comprising:
surgically implanting an angle supported anterior chamber ocular implant having an optic with desired refractive properties and a haptic having a proximal portion, a distal portion, and a transitional portion, wherein the proximal portion of the haptic resides substantially within the radial plane of this optic, wherein the distal portion of the haptic resides substantially in a radial plane parallel to the radial plane of the optic, wherein the transitional portion possesses a double reflex curvature, and
anchoring the distal portion of the haptic into the anatomic angle of the eye.

11. A method according to claim 10, when the distal portion of the haptic further comprises an extension pad radially extending and tapering in thickness from the haptic in a direction anterior to the surface of the optic, and wherein the extension pad is anchored into the anatomic angle.
